(19) United States
(12) Patent Application Publication
(10) Pub. No.: US 2008/0194015 A1
(43) Pub. Date: Aug. 14, 2008

(54) Algae mass-producing device having radiation source for hormesis

(75) Inventors: Yean-Chang Chen, Keelung City (TW); Meng-Chou Lee, Taipei City (TW); Ming-Chao Kuo, Hsinchu City (TW); Wen-Song Hwang, Longtan Shiang (TW); Chia-Chieh Chen, Taoyuan County (TW); Lee-Chung Men, Zhonghe City (TW); Bin Lin, Hualien City (TW); Kuan-Yin Chen, Xinzhuang City (TW); Hsueh-Hsuan Liu, Kaohsiung City (TW); Wei-Hsi Chen, Taipei City (TW)

Correspondence Address: TROXELL LAW OFFICE PLLC, 5205 LEESBURG PIKE, SUITE 1404, FALLS CHURCH, VA, 22041, US

(73) Assignee: ATOMIC ENERGY COUNCIL - INSTITUTE OF NUCLEAR ENERGY RESEARCH, Taoyuan (TW)

(21) Appl. No.: 11/704,377

(22) Filed: Feb. 9, 2007

(51) Int. Cl. C12M 1/42

(52) U.S. Cl. 435/292.1

(57) ABSTRACT

Algae are irradiated to obtain a hormesis so that its thickness is increased or its growth time is shortened. A cubic rack with an adhesion unit is used so that more algae are planted to be cultured on the adhesion unit through the irradiation for mass production.

### FIELD OF THE INVENTION

The present invention relates to mass-producing algae; more particularly, relates to processing algae through a hormesis by irradiation to grow thicker or to shorten a growth time for mass-production.

### DESCRIPTION OF THE RELATED ART

A general method for culturing algae uses a flat rack 8 winded with a cotton rope 81 (as shown in FIG. 10). A plurality of algae spores 82 are planted on the cotton rope 81 (as shown in FIG. 11). Then the flat rack 8 is put into a petri dish 9 having a sea water 91 (as shown in FIG. 12). After the algae spores 82 have been grown into fronds 83 in a required time (as shown in FIG. 12), the algae is taken off.

Although algae spores 82 are grown into fronds 83 in a required time in the above prior art, algae spores 82 are only planted on the cotton rope 81 on a surface of the flat rack 8 because the other surface of the flat rack 8 are closely contacted with the petri dish 9; not to mention a small size of the flat rack 8. Therefore, algae spores 82 are grown into fronds 83 only on the cotton rope 81 of the surface of the flat rack 8; and algae are not massed produced. Hence, the prior art does not fulfill users' requests on actual use.

### SUMMARY OF THE INVENTION

The main purpose of the present invention is to process algae with a hormesis by irradiation to grow thicker or to shorten a growth time for mass-production.

To achieve the above purpose, the present invention is an algae mass-producing device having an ionizing radiation source, comprising a container having a containing space and a filter; a cubic rack in the containing space; an adhesion unit winding around the cubic rack; and an irradiation unit, where, producing algae by using the present invention comprises steps of: (a) obtaining the cubic rack; (b) filling the containing space with a culture solution and putting the cubic rack in to the culture solution; (c) planting algae spores to be cultured in the containing space through a flow-through culturing with the filter; (d) irradiating a required dose to the algae spores by the irradiation unit; (e) after the algae spores are cultured into algae seedlings, taking out the adhesion unit to an outdoor flowing-water pool to be cultured again and, (f) when the algae grow to a required length, the algae are taken off from the adhesion unit to be cleaned and dried. Accordingly, a novel algae mass-producing device having an ionizing radiation source is obtained.

### BRIEF DESCRIPTIONS OF THE DRAWINGS

The present invention will be better understood from the following detailed description of the preferred embodiment according to the present invention, taken in conjunction with the accompanying drawings, in which

FIG. 1 is the perspective view showing the preferred embodiment according to the present invention;

FIG. 2 is the explosive view showing the preferred embodiment;

FIG. 3 is the flow view showing the algae mass-producing method;

FIG. 4 is the view showing obtaining the cubic rack;

FIG. 5 is the view showing putting into the containing space;

FIG. 6 is the view showing planting the algae to be cultured;

FIG. 7 is the view showing irradiating the algae;

FIG. 8 is the view showing culturing the algae again;

FIG. 9 is the view showing taking off the algae to be cleaned and dried; and

FIG. 10 to FIG. 13 are the views of the prior art.

### DESCRIPTION OF THE PREFERRED EMBODIMENT

The following description of the preferred embodiment is provided to understand the features and the structures of the present invention.

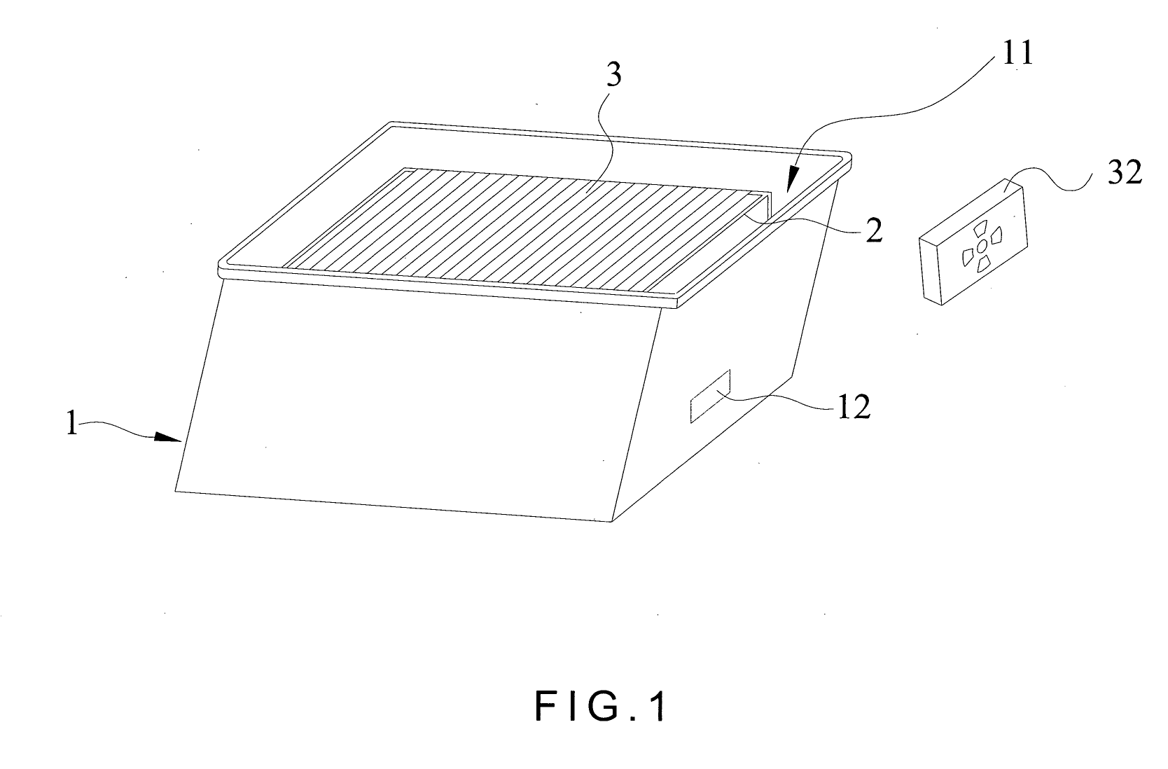

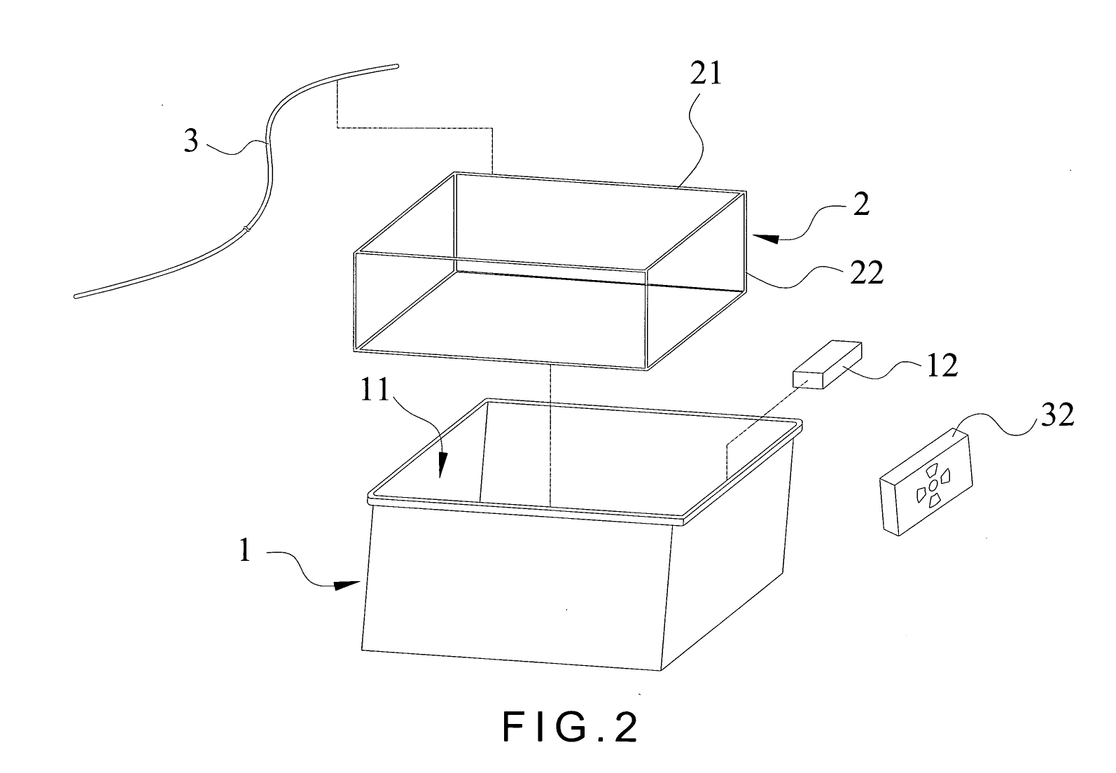

Please refer to FIG. 1 and FIG. 2, which are a perspective view and an explosive view showing a preferred embodiment according to the present invention. As shown in the figures, the present invention is an algae mass-producing device having an ionizing radiation source, comprising a container 1, a cubic rack 2, a n adhesion unit 3 and an irradiation unit 32, where algae is processed through a hormesis by irradiation to grow thicker or to shorten a growth time for mass-production.

The container 1 has a containing space 11; and, the containing space 11 has a filter 12.

The cubic rack 2 is put in to the containing space 11; and the cubic rack 2 comprises a plurality of transverse rods 21 and straight rods 22.

The adhesion unit 3 is winded around the cubic rack 2; and the adhesion unit 3 is a cotton rope.

The irradiation unit 32 is an ionizing radiation source. Thus, with the above structure, a novel algae mass-producing device having an ionizing radiation source is obtained.

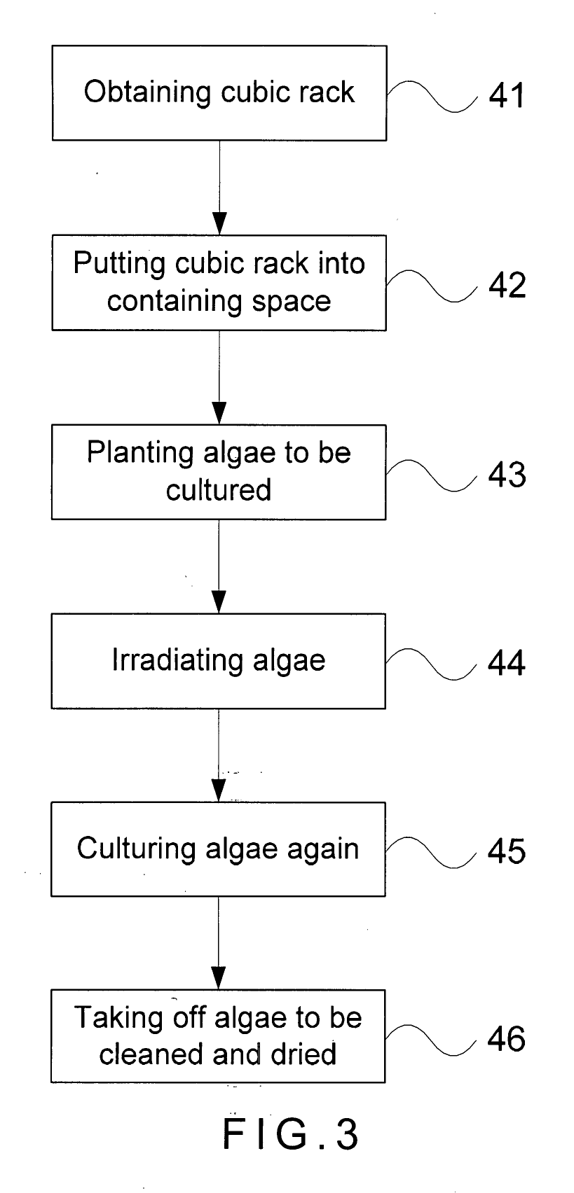

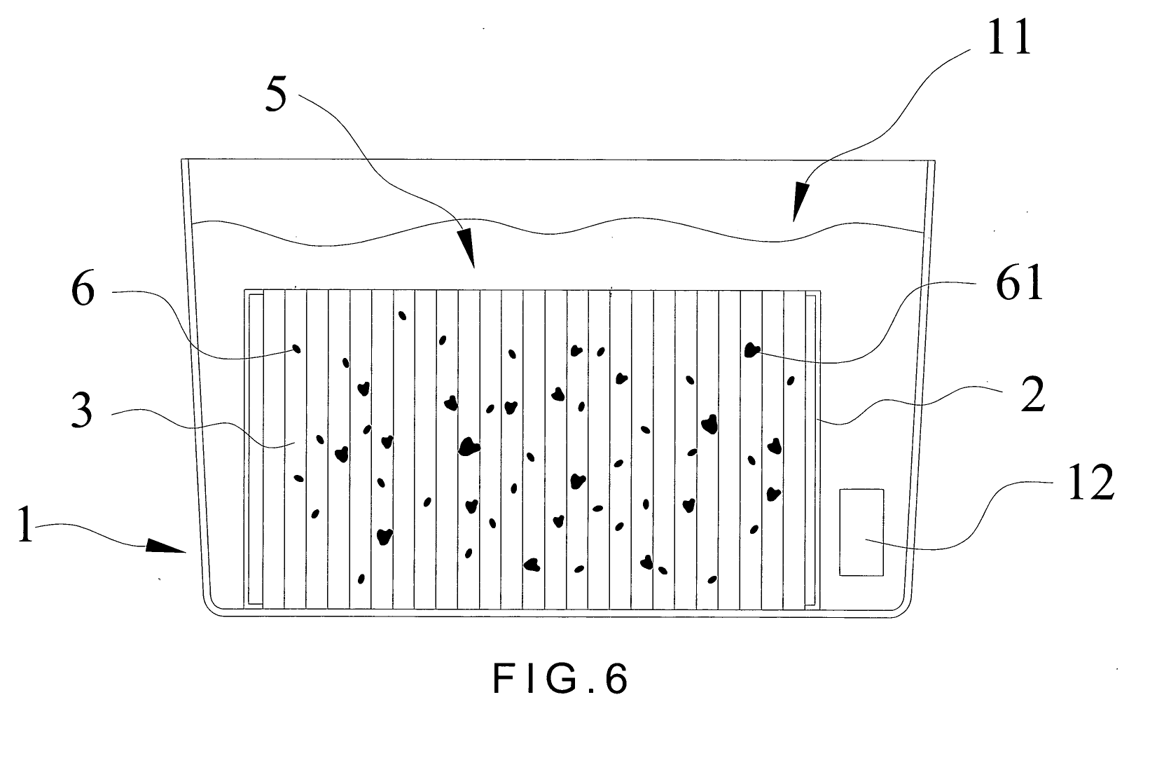

Please refer to FIG. 3 to FIG. 9, which are a flow view showing an algae mass-producing method; and views showing obtaining a cubic rack, putting the cubic rack into a containing space, planting algae to be cultured, irradiating the algae, culturing the algae again, and taking off the algae to be cleaned and dried. As shown in the figures, algae is mass-produced by the present invention, comprising the following steps:

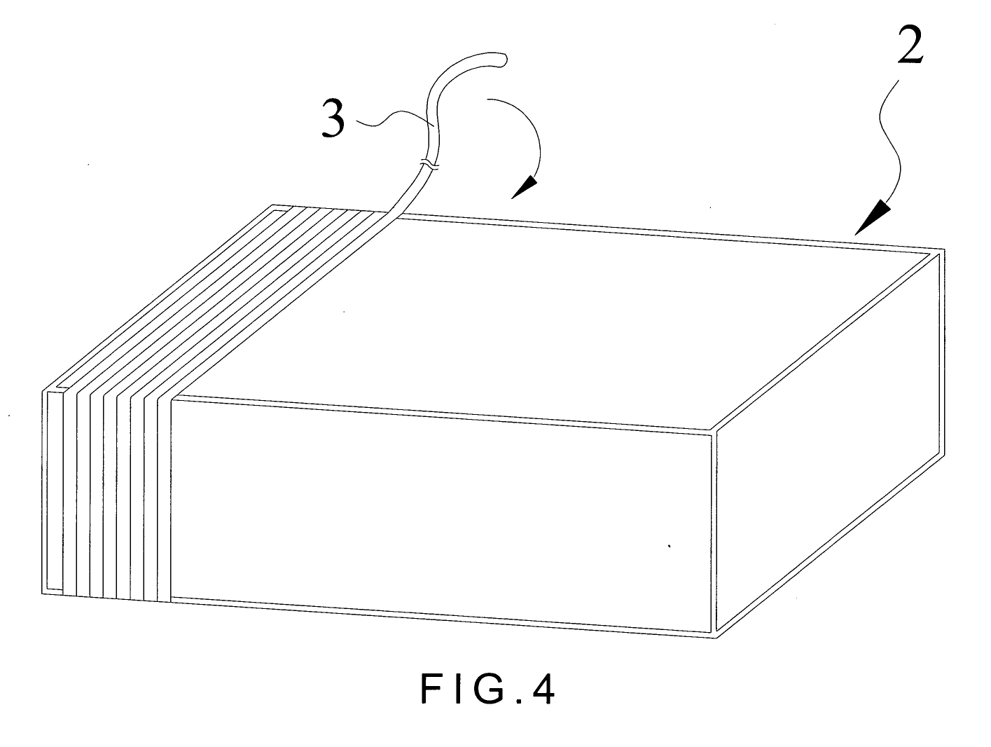

(a) Obtaining cubic rack 41: As shown in FIG. 4, a cubic rack 2 is obtained, which has a length of 40 centimeters (cm), a width of 30 cm and a height of 30 cm; and is made of a stainless steel having a diameter of 0.7 cm. The cubic rack 2 is winded with an adhesion unit 3, where the adhesion unit 3 is a cotton rope having a 1 cm diameter and a length of 33 meters (m).

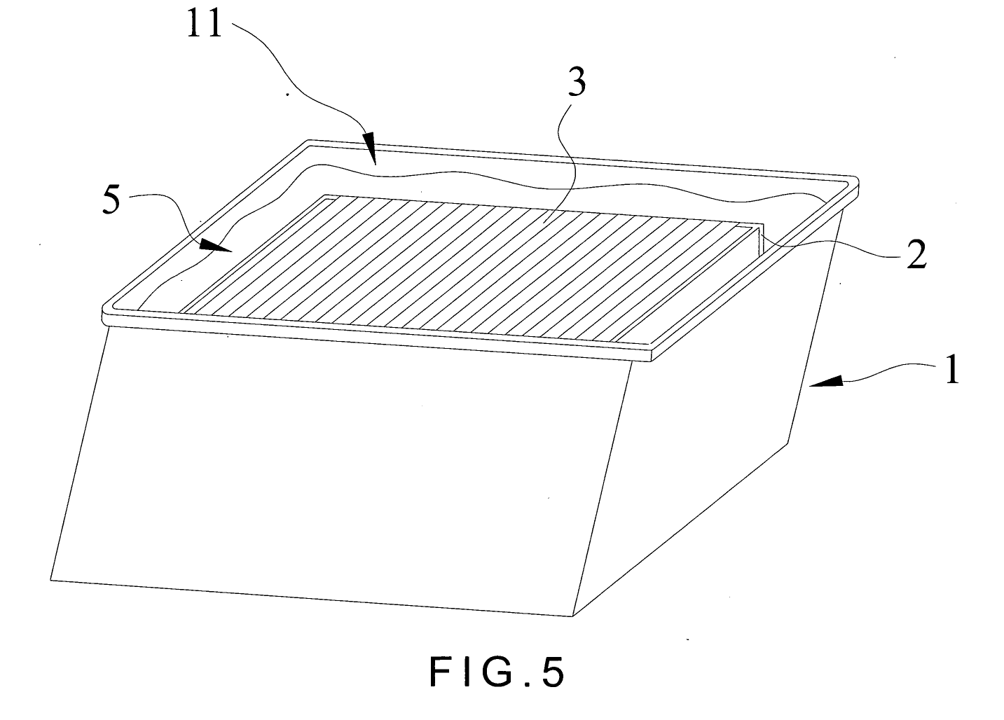

(b) Putting cubic rack in to containing space 42: As shown in FIG. 5, the cubic rack 2 winded with the adhesion unit 3 is put into a containing space 11 of a container 1, where the container 1 is a 120-liter rectangle tub of polyvinyl chloride (PVC); a culture solution 5 having 100 liters is filled in the containing space 11; the culture solution 5 is a sea water; and the water level of the culture solution 5 is higher than the upper level the adhesion unit 3.

(c) Planting algae to be cultured 43: A plurality of algae spores 6 is planted on the adhesion unit 3; and the containing space 11 is set with a filter 12. Then the container 1 is put under the sun (in a green house) for culturing, where zoospores among the algae spores 6 are completely adhered on the adhesion unit 3 within one to two days; and visible algae seedlings 61 are seen in the fifteenth day; and, when fronds are obtained with a length of 0.5 to 1.0 millimeters (mm), the filter 12 is used to filter sea water in a speed of 50 to 75 milliliters per second (ml/sec) for processing a flow-through culturing to the algae spores 6.

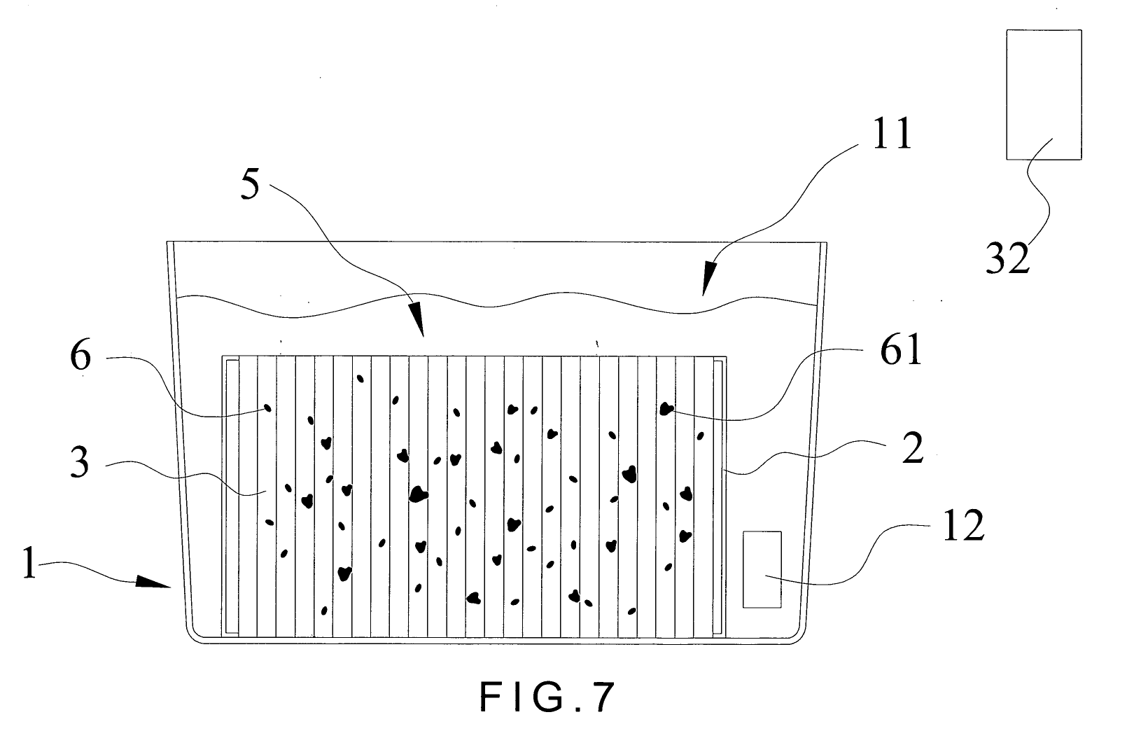

(d) Irradiating algae 44: As shown in FIG. 7, the algae spores 6 are irradiated with a required dose by an irradiation unit 32 of an ionizing radiation source, where the required dose irradiated by the irradiation unit 32 is controlled by checking the time spent in irradiating the algae. Furthermore, the irradiation is finished with the following steps: (i) the algae are put into a watertight box closed with a cover; (ii) the watertight box is sunk to an irradiating seat along a track in a water pool; (iii) an irradiating source is put at a side of the irradiating seat; (iv) the watertight box is irradiated at a rate of 5 grays per minute for 1, 2 or 3 minutes; and, (v) time is checked by a stop watch so that, when the time is up, the watertight box is moved away from the irradiating source or even the water pool to obtain a dose of 5, 10 or 15 grays.

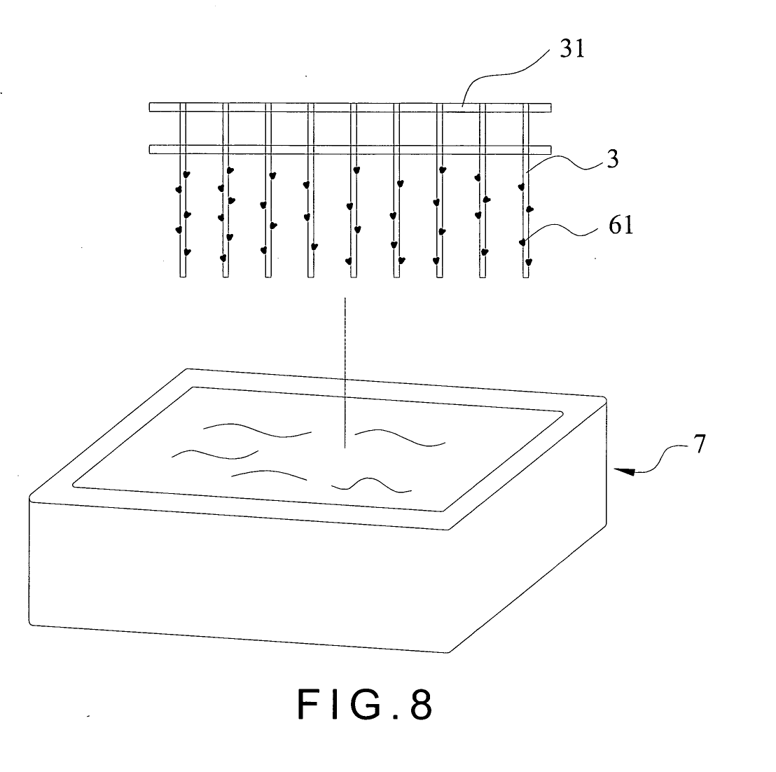

(e) Culturing algae again 45: As shown in FIG. 8, after the algae spores 6 on the adhesion unit 3 are cultured into algae seedlings 61 after 40 days, the adhesion unit 3 with the algae seedlings 61 having a length about 3.0 to 5.0 mm is taken off. The adhesion unit 3 are cut into 100 cm sections; and the sections are cross-linked with a rope 31 having a 0.6 cm diameter. Then the adhesion unit 3 is moved to an outdoor flowing-water pool 7 to be cultured again with a sea water, where the flowing-water pool 7 has a length of 4.4 m, a width of 2.2 m, a height of 1.3 m, a water depth of 0.9 m and a sea water volume of 8.7 cubic meter; and the sea water is flown in a speed of 300 ml/sec.

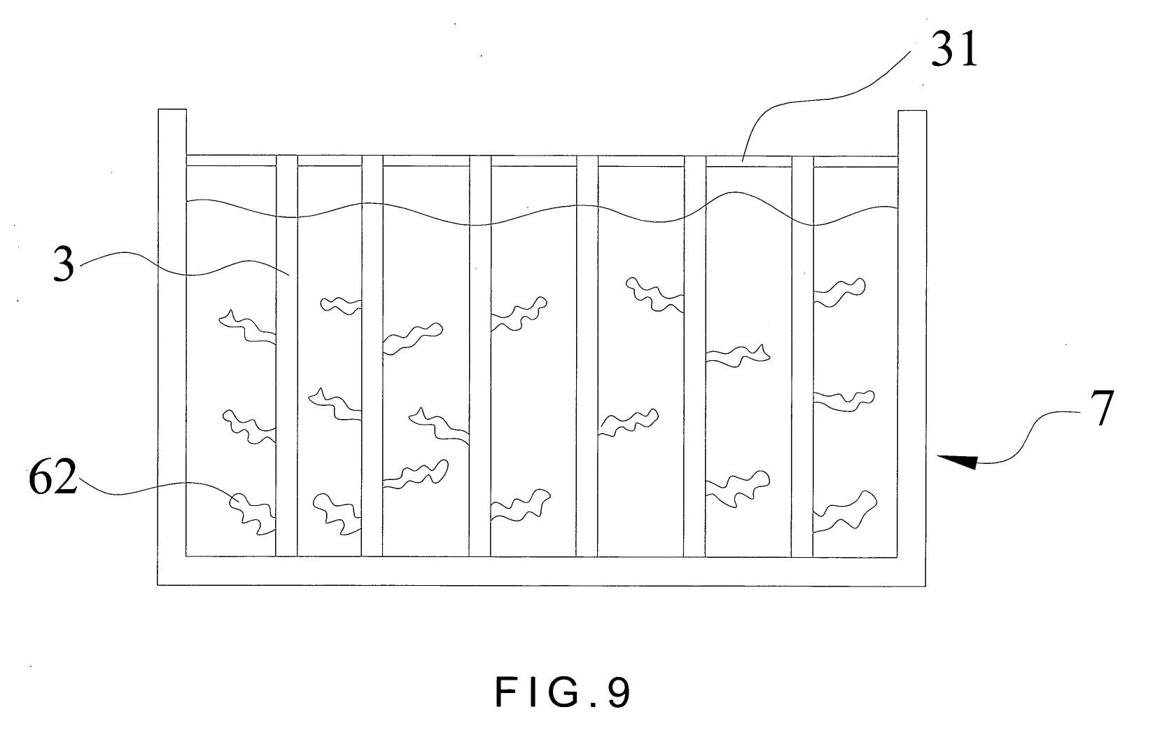

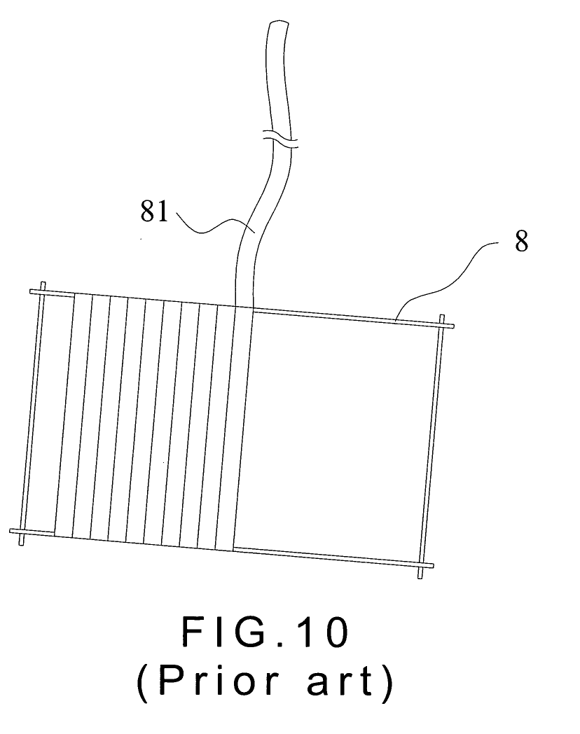

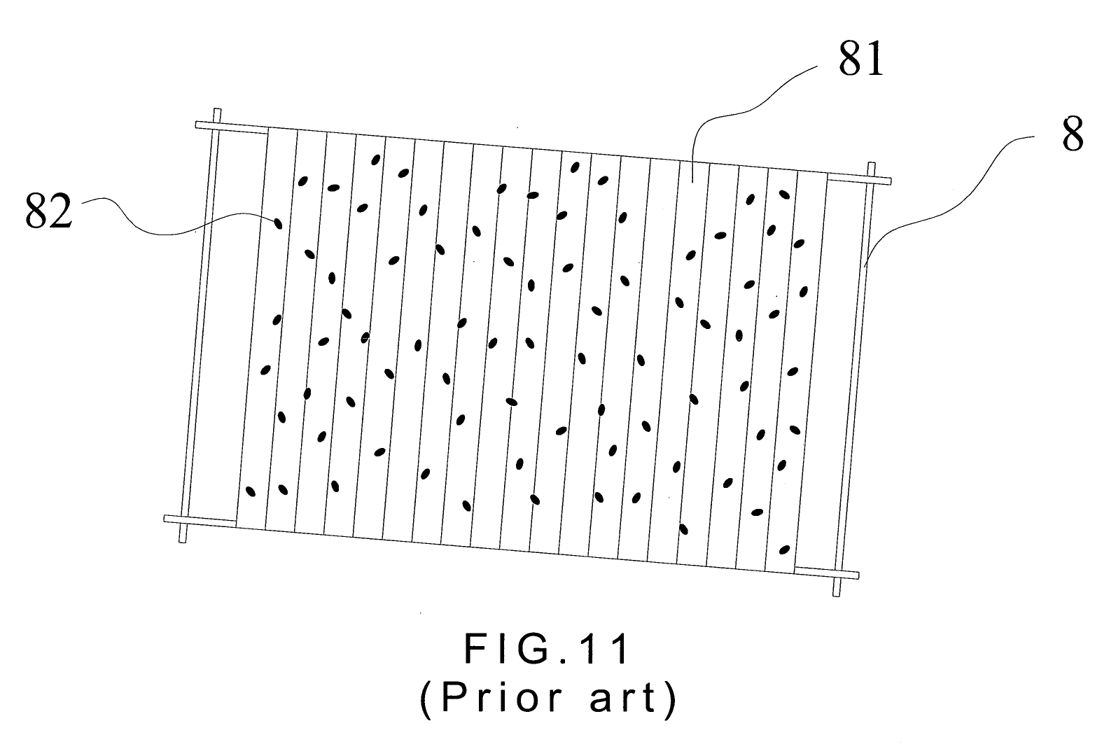

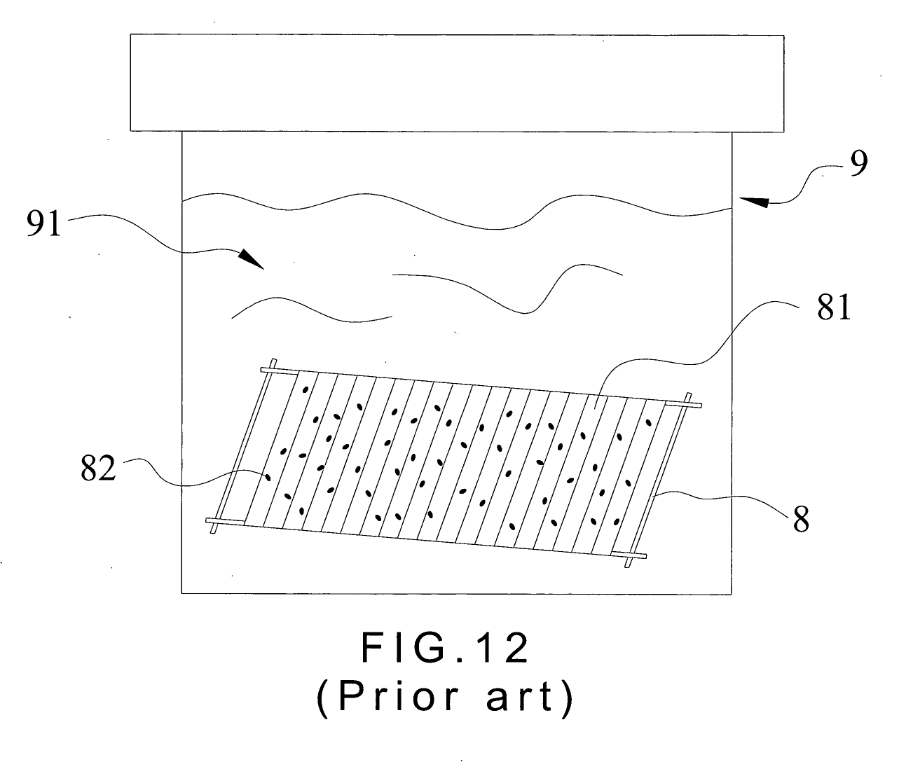

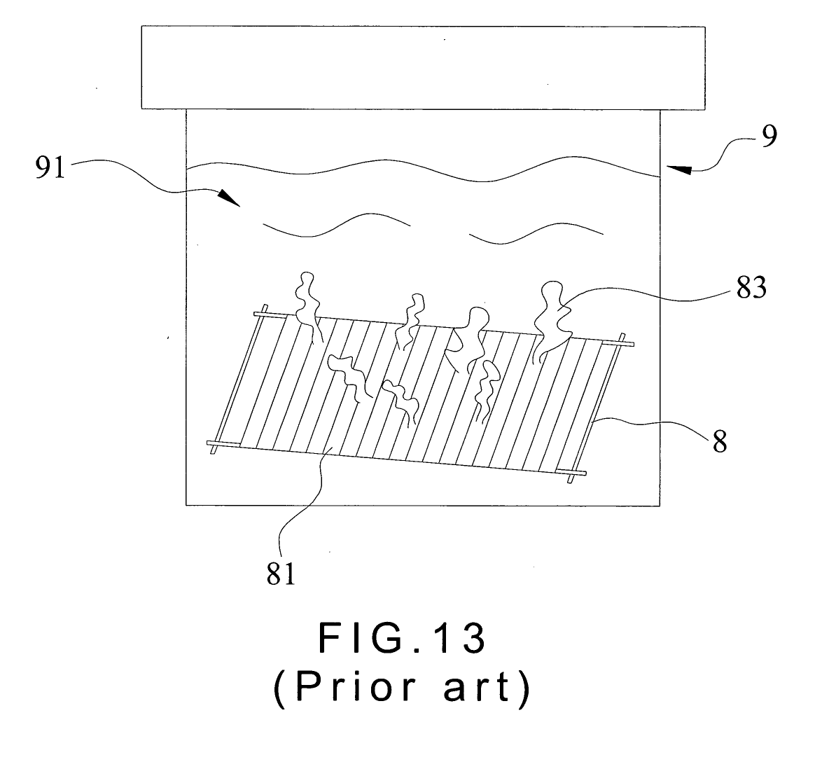

(f) Taking off algae to be cleaned and dried 46: As shown in FIG. 9, when the algae have grown into a frond 62 having a required length (about more than 10 cm), the algae 52 are taken off from the adhesion unit 3 directly. After the algae 52 are washed with a filtered sea water, the algae 52 are put into an oven at 70 Celsius degrees to be dried.

To sum up, the present invention is an algae mass-producing device having an ionizing radiation source, where algae is processed through a hormesis by irradiation to grow thicker or to shorten a growth time for mass-production.

The preferred embodiment herein disclosed is not intended to unnecessarily limit the scope of the invention. Therefore, simple modifications or variations belonging to the equivalent of the scope of the claims and the instructions disclosed herein for a patent are all within the scope of the present invention.

What is claimed is:

1. An algae mass-producing device having an ionizing radiation source, comprising:
a container, said container having a containing space, said containing space having a filter;
a cubic rack, said cubic rack being deposed in said containing space;
an adhesion unit, said adhesion unit being winded around said cubic rack; and
an irradiation unit, said irradiation unit being an ionizing radiation source irradiating on algae.

2. The device according to claim 1,
where in said containing space is filled with a culture solution.

3. The device according to claim 2,
wherein said culture solution is a sea water.

4. The device according to claim 1,
where in said cubic rack comprises a plurality of transverse rods and straight rods.

5. The device according to claim 1,
wherein said adhesion unit is planted with a plurality of algae spores.

6. The device according to claim 1,
where in said adhesion unit is a cotton rope.

7. The device according to claim 1,
wherein said device has an algae mass-producing method, comprising
steps of:
(a) obtaining said cubic rack, said cubic rack being winded with said adhesion unit;
(b) putting said cubic rack into said container, said container being filled with a culture solution;
(c) planting a plurality of algae spores on said adhesion unit, and processing a flow-through culturing to said adhesion unit, and said filter in said container;
(d) irradiating a dose to said algae spores by said irradiation unit;
e) after said algae spores being grown into algae seedlings, taking off said adhesion unit to process said algae seedlings with a flow-through culturing in a flowing-water pool; and
(f) after growing said algae to a length, taking off said algae from said adhesion unit to be cleaned and dried.
